(19) United States
(12) Patent Application Publication
(10) Pub. No.: US 2019/0142736 A1
(43) Pub. Date: May 16, 2019

(54) Thermal Protectant For Hair

(71) Applicant: Mantrose-Haeuser Co., Inc., Westport, CT (US)

(72) Inventors: Stephen A. Santos, Cumberland, RI (US); Bin Zhong, North Attleboro, MA (US)

(21) Appl. No.: 16/174,863

(22) Filed: Oct. 30, 2018

Related U.S. Application Data
(60) Provisional application No. 62/586,502, filed on Nov. 15, 2017.

(51) Int. Cl. A61K 8/98; A61Q 5/06; A61K 8/06; A61K 8/04

(52) U.S. Cl. CPC A61K 8/987; A61Q 5/06; A61K 2800/24; A61K 8/046; A61K 8/06

(57) ABSTRACT

Shellac is used as a thermal protectant to protect hair from damage by the heat generated from styling tools such as flat irons, curling irons, curling brushes, blow dryers and the like.

### RELATED APPLICATIONS

This application claims priority to and any benefit of U.S. Provisional Patent Application No. 62/586,502, filed Nov. 15, 2017, the content of which is incorporated herein by reference in its entirety.

### BACKGROUND

Earlier hair styles called for curly hair having lots of hair volume and body. Products that were designed to meet this need were chemically active. So, in addition to changing hair configuration to make it curly, they also did extensive chemical damage.

Styles have now shifted to long straight hair that has movement, bounce and shine. To meet this need, hair straightening products have been introduced such as Brazilian hair straighteners which transform unruly hair to straight hair with lots of shine and manageability. Unfortunately, these products normally contain high levels of formaldehyde, which can lead to chemical damage.

Hair care manufacturers have responded to this problem by recommending that hot styling tools such as flat irons, curling irons, curling brushes, blow dryers, etc., be used to straighten hair. While this may seem simpler than chemical treatments, it also comes with disadvantages, namely physical damage to the hair.

Hair is composed of an inner medulla which is surrounded by a cortex layer that is protected by several layers of cuticles. Cuticles are like shingles on a roof that protect the cortex from the elements. When hair is repeatedly exposed to heat, the cuticles are gradually degraded, exposing the cortex layer. As a result, the hair not only is prone to developing split ends but also can become frizzy and unmanageable especially in hot and humid weather.

Since this type of physical damage is basically irreversible, thermal protectant products have been formulated to prevent such damage from occurring in the first place. These products are usually composed of one or more polymers which have low glass transition temperatures and, hence, are capable of forming uniform layers on the hair surfaces. Especially desirable polymers are those which not only can deflect heat, but also are aesthetically pleasing to the touch, avoid causing any build up on hair and can be easily removed with conventional shampoos.

Normally, these products are applied in spray form, as this is most convenient. However, they can also be applied from a foaming product such as a hair mousse. The hair is then normally combed to get a uniform layer of the protectant on the surfaces of the hair cuticles, after which the hair is styled using a flat iron, curling iron, curling brush or blow dryer.

### SUMMARY

In accordance with this invention, it has been found that shellac can function as an especially effective hair protectant.

Accordingly, this invention provides an improvement in processes for styling hair in which a thermal protectant capable of protecting the hair from damage caused by the application of heat is applied to the hair following which heat is applied to the hair to achieve the desired styling, the improvement wherein the thermal protectant is shellac.

### DETAILED DESCRIPTION

### Basis Weight

Unless otherwise indicated, the concentrations of ingredients specified below are given in terms of the weight of the ingredient based on the weight of the thermal protectant compositions of this invention as a whole but excluding any propellant that might be present. That is to say, in those embodiments of this invention in which the thermal protectant is provided in the form of a sprayable composition in an aerosol container, the concentration of ingredients of this composition, as described below, will be understood to exclude any propellant that may also be present in the container.

### Sprayable and Non-Sprayable Compositions

The thermal protectant compositions of this inventions can be provided in the form of non-sprayable compositions, i.e., compositions which are too viscous to be conveniently sprayed from conventional mechanical pump sprayers or conventional pressurized aerosol canisters. Specific examples include lotions, pastes, creams and gels.

More commonly, however, the thermal protectant compositions of this inventions will take the form of sprayable compositions which are formulated for being delivered by means of conventional pressurized aerosol canisters using a propellant, conventional mechanical pump sprayers or both. In this regard, see U.S. Pat. No. 6,126,930, the disclosure of which is incorporated by reference. As well appreciated in the art, a wide range of mechanical pump spray devices and aerosol canister systems are well known. In addition, a wide range of propellant materials are also well known for use in these canister systems. Nonlimiting examples include lower molecular weight hydrocarbons such as propane, n-butane and isobutane, nitrogen, carbon dioxide, nitrous oxide, and so forth. Mixtures of such propellants can also be used.

If a propellant is used, the amount may be as little as about 10 wt. % to as much as about 75% propellant, based on the weight of the composition as a whole including the propellant. Propellant concentrations on the order of about 20 wt. % to about 50 wt. % are more typical.

### Alcohol Based Compositions

In some embodiments of this invention, the thermal protectant compositions of this invention are alcohol-based. In this context, “alcohol-based” means that at least 50 wt. % of the liquid carrier of the composition, based on the weight of the liquid carrier, is composed of one or more alcohols. Normally, these compositions contain at least about 40 wt. %, at least about 50 wt. % or even at least about 55 wt. % alcohol, based on the weight of the composition as a whole, excluding propellant. In addition, these compositions typically contain no more than about 95 wt. %, no more than about 85 wt. %, no more than about 80 wt. %, or even no more than about 75 wt. %, alcohol, based on the weight of the composition as a whole, excluding propellant. Normally, all of the shellac of these compositions is dissolved in its liquid carrier, although this is not necessary.

Any alcohol which has previously been used, or which may be used in the future, as a liquid carrier in a sprayable, topical pharmaceutical composition can be used as the alcohol in the thermal protectant compositions of this inventions. Thus, monohydric alcohols having 2 to 8 carbon atoms can be used. Specific examples include ethanol, n-propanol, iso-propanol, and mixtures thereof. Ethanol is preferred, especially denatured ethanol including any known cosmetically or pharmaceutically-acceptable denaturing agent. Specific commercially available alcohols which are useful for this purpose include SD (Specially Denatured) Alcohol 3-A, SD Alcohol 30, SD Alcohol 39, SD Alcohol 39-B, SD Alcohol 39-C, SD Alcohol 40, SD Alcohol 40-B, SD Alcohol 40-C. As well known, these designations have been assigned by the U.S. Bureau of Alcohol, Tobacco and Firearms to refer to denatured ethanol compositions containing different amounts and types of common denaturants including denatonium benzoate, quassin, brucine and brucine sulfate.

The alcohol-based thermal protectant compositions of this invention may also be essentially free of water, although this is not required. “Essentially free of water” means that the amount of water in these composition is no more than is contributed by the 190 proof alcohol that is used to make the composition, i.e., a maximum water content of no more than about 2 wt. %. Maximum water contents of no more than about 1.0 wt. %, no more than about 0.5 wt. %, and even no more than about 0.1 wt. % are contemplated in these embodiments.

### Water-Based Compositions

In other embodiments of this invention, the thermal protectant compositions of this invention are water-based. In this context, “water-based” means that a significant majority of the carrier liquid forming the composition is water. These water-based thermal protectant compositions can take the form of, a solution in which the shellac is dissolved in the aqueous carrier liquid of the composition. In addition, they can also take the form of an oil-in-water emulsion in which the shellac is emulsified in the aqueous carrier liquid of the composition.

These water-based thermal protectant compositions differ from their alcohol-based counterparts in that they do not contain the large amounts of alcohol found in their alcohol-based counterparts. For example, when these water-based compositions are in the form of shellac solutions, they can contain no more than about 20 wt. %, more desirably no more than about 15 wt. % or even no more than about 10 wt. % alcohol, based on the weight of the thermal protectant compositions of this invention as whole, excluding any propellant that might be present if any. Meanwhile, when these water-based compositions are in the form of shellac emulsions, they can contain up to about 11 wt. %, more desirably up to about 5 wt. %, up to about 3 wt. % or even up to about 1 wt. % alcohol, based on the weight of the thermal protectant compositions of this invention as whole, excluding any propellant that might be present if any. Alcohols may promote drying of the skin and scalp, which is avoided in these water-based compositions, since alcohols are essentially absent or at least present in only small amounts.

The thermal protectant compositions of this invention, when in the form of a solution of shellac dissolved in its aqueous carrier liquid, differ from their alcohol-based counterparts for the additional reason that the protective films formed by these shellac solutions exhibit a different tactile sensation or “feel” than that provided by their alcohol-based counterparts. This is because the shellac in these compositions is water-sensitive, the result of which is that the protective films formed this type of shellac can “breath” in the sense of not blocking moisture evaporating from the scalp. Protective films formed from shellac which is dissolved in alcohol, or which is emulsified in water, are generally impervious to skin moisture since they are made from water-insoluble shellac.

The thermal protectant compositions of this invention when in the form of aqueous shellac solutions also differ from the other thermal protectant compositions of this invention in terms of the nature of the shellac from which they are made.

As well understood in the art, shellac is readily soluble in alcohol as well as water with an alkaline pH. However, it is essentially insoluble in water of neutral or acidic pH. Therefore, to make the thermal protectant compositions of this invention in the form of an aqueous shellac solution, the shellac must first be treated to make it water-soluble.

Once way this can be done is by combing solid shellac (e.g., powder or flakes) with saponified water containing a small but suitable amount of alcohol, followed by heating and vigorous mixing of the composition so formed until the shellac is completely dissolved.

Another way this can be done is by dissolving the solid shellac into a small amount of alcohol and then combining the shellac solution so formed with a similar amount of alkaline water, with stirring. In this context, a “similar” amount of water means that the weight ratio of the shellac solution to alkaline water is desirably between about 0.75:1 to 2:1, more desirably between about 1:1 to 1.7:1, or even about 1.25:1 to 1.5:1.

For example, solid, flake shellac can be slowly sprinkled into approximately twice as much ethanol on a weight basis heated to 75-80° C. with stirring. Once the shellac is completely dissolved, the shellac solution so formed can then be added to approximately the same amount or slightly less of alkaline water at a pH of about 7.5-8, also maintained at about 75-80° C. with stirring. The composition so formed is then stirred until an aqueous solution containing the shellac and alcohol is obtained. Thereafter, the composition can be allowed to cool to about 50 to 60° C., after which it is then combined with the remaining ingredients of the composition, which have already been previously combined. If necessary, the pH of the final composition can be further adjusted to reach a final target pH of about 7.5 to 8.

The amount of shellac in these compositions, i.e., the thermal protectant compositions of this invention when in the form of a shellac solution, will normally be about 1-20 wt. %, based on the weight of the composition as a whole, excluding any propellant that might be present as mentioned above. Desirably, the amount of shellac dissolved in these compositions will be about 2 to 15 wt. %, about 3 to 10 wt. % or even 4 to 7 wt. %.

Meanwhile, the amount of carrier liquid in the thermal protectant compositions of this invention, whether alcohol-based or water-based, can vary widely and depends, among other things, on the form of the composition to be made. That is to say, the amount of carrier liquid in these compositions depends, among other things, on whether the composition is to be made in the form of a lotion, cream, gel, foam or sprayable composition.

For example, the thermal protectant compositions of this invention, when made in the form of a lotion, will typically contain about 45 to 80 wt. %, more typically about 50 to 70 wt. %, or even about 55 to 65 wt. % carrier liquid. When made in other forms, the amount of carrier liquid to include can be easily determined using routine experimentation.

### Other Conventional Ingredients

In addition to shellac and carrier liquid, the thermal protectant compositions of this invention can also contain any other ingredient which is conventionally included in similar compositions. Examples include stabilizers, emulsifiers, rheology control agents, moisturizers, emollients, hair conditions, perfumes, astringent salts, antioxidants, preservatives, and so forth.

### Shellac Film Former

In accordance with this invention, shellac is used as the film former in the thermal protectant composition of this invention. Preferably, a dewaxed shellac and in particular a bleached dewaxed shellac is used.

Shellac is a naturally occurring thermoplastic obtained from secretions of the female lac bug. It exhibits a remarkable combination of properties including low permeabilities to oxygen, water vapor, CO2, ethylene and various odors, low lipid solubility, excellent color and excellent clarity.

Shellac is obtained from seedlac, an insect secretion, by removing debris from the seedlac and then further processing the seedlac to obtain the desired product. Commercially, shellac is available in two different types, bleached shellac and orange shellac. Moreover, both of these shellacs are available in refined (i.e., dewaxed) as well as unrefined (regular) versions. In addition, each of these four different varieties of shellac are available in different physical forms, e.g., solid flakes and aqueous and/or alcohol solutions. In addition, some of these different varieties are also available in different grades. For example, dewaxed orange shellac is available in a variety of different grades ranging from faint orange to intense orangish red.

As described in U.S. Pat. No. 6,348,217, the entire disclosure of which is incorporated herein by reference, bleached shellac is made by dissolving seedlac in aqueous alkali and then adding a bleaching agent such as sodium hypochlorite. The product so obtained is then precipitated and dried to produce regular bleached shellac. Alternatively, the dissolved bleached shellac can be refined by known techniques to remove its wax content before precipitating and drying, thereby producing dewaxed bleached shellac. In contrast, regular orange shellac is made by melting seedlac, sieving out the insolubles and then solidifying and flaking the product so obtained. Meanwhile, dewaxed orange shellac is made by dissolving the seedlac in alcohol, straining out the insolubles, filtering out wax particles and passing the solution so obtained through activated carbon to decolorize before solidifying and flaking.

In accordance with this invention, each of these different types of shellac can be used as the film forming polymer in the thermal protectant composition of this invention. Shellac is readily soluble in alcohol, especially ethanol, and so it easily forms sprayable compositions with the other ingredients found in a typical sprayable composition. However, because shellac is naturally-occurring, it is readily biodegradable in contrast to the acrylic and acrylamide polymers which are currently used as film formers in many sprayable topical compositions. In addition, shellac is less expensive than these synthetic polymers.

Regardless of the form of the thermal protectant compositions of this invention, the amount of shellac film former in these compositions can be as little as 0.5 wt. % or less based on the weight of the entire composition as a whole but excluding any propellant that might be present, although minimum shellac concentrations of about 1.0 wt. %, about 1.5 wt. % or even 2.0 wt. % are more common. Similarly, the thermal protectant composition of this invention can contain as much as 10 wt. % or more shellac, although maximum shellac concentrations of about 7.0 wt. %, about 5.0 wt. % or even 3.0 wt. % are more common.

### Method of Use

The thermal protectant compositions of this invention are used in the same way as conventional hair thermal protectants, i.e., by applying the protectant composition to the hair preferably before the application of heat, optionally and preferably combing or otherwise treating the hair to distribute the thermal protectant uniformly, and they applying heat for styling the hair as desired, typically by using a flat iron, curling iron, curling brush, blow dryer or similar heat-generating styling tool.

Although only a few embodiments of this invention have been described above, it should be appreciated that many modifications can be made without departing from the spirit and scope of this invention. All such modifications are intended to be included within the scope of this invention, which is to be limited only by the following claims:

1. An improvement in processes for styling hair in which a thermal protectant capable of protecting the hair from damage caused by the application of heat is applied to the hair following which heat is applied to the hair to achieve the desired styling, the improvement wherein the thermal protectant is shellac.

2. The improvement of claim 1, wherein the thermal protectant comprises shellac and a carrier liquid.

3. The improvement of claim 3, wherein the thermal protectant is alcohol-based.

4. The improvement of claim 1, wherein the thermal protectant is water-based.

5. The improvement of claim 4, wherein the thermal protectant is a solution of a water-soluble shellac in an aqueous carrier liquid.

6. The improvement of claim 4, wherein the thermal protectant is an oil-in-water emulsion of shellac emulsified in an aqueous carrier liquid.

7. The improvement of claim 1, wherein the thermal protectant is in the form of a sprayable composition.

8. The improvement of claim 1, wherein the thermal protectant is in the form of a foam.

9. The improvement of claim 1, wherein the wherein the shellac is dewaxed shellac.

10. The improvement of claim 9, wherein the shellac is dewaxed bleached shellac.

11. The improvement of claim 1, wherein heat is applied to the hair by at least one of a flat iron, curling iron, curling brush and blow dryer.
